(19) United States
(12) Patent Application Publication
(10) Pub. No.: US 2021/0386530 A1
(43) Pub. Date: Dec. 16, 2021

(54) INTERDENTAL DEVICE

(71) Applicant: Koorosh Banayan, Encino, CA (US)

(72) Inventor: Koorosh Banayan, Encino, CA (US)

(21) Appl. No.: 16/900,166

(22) Filed: Jun. 12, 2020

(51) Int. Cl. A61C 15/02; A46B 11/00; A46B 9/04; A61K 8/02; A61Q 11/00

(52) U.S. Cl. CPC A61C 15/02; A46B 11/0003; A46B 9/04; A61C 2202/00; A61Q 11/00; A46B 2200/108; A61K 8/0204

(57) ABSTRACT

A single-use oral delivery device includes a housing unit configured to house a predetermined amount of oral medication, vitamins and or minerals to be applied to an interdental space of a user, and an interdental device configured to be interlocked with the housing unit, wherein the interdental device includes a cap at a top portion for interlocking and sealing with the housing unit, and an applicator tip at a bottom portion for applying oral medication, vitamins or minerals to the interdental space.

### TECHNICAL FIELD

The present disclosure relates to interdental and oral delivery devices and methods for manufacturing the same, and more particularly to devices for cleaning, massaging, and treating surfaces of teeth and gums including interproximal and subgingival areas, and applying predetermined amounts of vitamins, mineral and/or pharmaceutical compounds to such areas.

### BACKGROUND

Oral application for buccal delivery is ideal for delivering drugs, minerals, vitamins, and other forms of active agents, within or through the buccal mucosa in order to achieve a local or systemic effect. This route is particularly attractive because materials can be lodged directly into the gum, the interdental cavity, and interproximal and subgingival areas, and the substances absorbed through the buccal mucosa bypass gastrointestinal enzymatic degradation and the hepatic first-pass effect. The mouth has a relatively large area for drug/active agent application and good accessibility as compared to other routes. In addition, the mucosa is resistant to damage or irritation because of the rapid cell turnover and frequent exposure to food. Examples of active agents include, but are not limited to, numbing agents, agents for sensitive teeth, and other treatments or medication for the treatment of localized or systemic ailments or conditions.

However, there is currently no effective means for the delivery of these materials to the desired site for localized or systemic use. Existing multi-use containers and applicators for oral delivery includes a dual chamber wherein antiseptic materials can be housed. However, such containers are difficult to fabricate, difficult to fill with different materials and are not cost effective. Also, the user does not know the quantity of active ingredients to be applied for each use. Further, users are reluctant to use the same applicator tip, over and over again to be put back into a container, only to be used again. Furthermore, multiple use of same applicator tip, may inevitably result in some material from the mouth to finding its way to the container, which over time can cause various bacteria and other infectious substances to seep into the material to be delivered thereby contaminating same.

### SUMMARY

One aspect of the present disclosure is to provide a single-use oral delivery device that includes a housing unit configured to house a predetermined amount of a material to be applied to an interdental space of a user; and an interdental device configured to be interlocked with the housing unit, where the interdental device includes a cap at a top portion configured to interlock and seal with the housing unit, and an applicator tip at a bottom portion configured to apply the predetermined amount of the material to the interdental space.

In another aspect of the present disclosure, an integrated pack of single-use oral delivery devices includes a plurality of housing units containing a predetermined amount of a material, and a plurality of interdental devices inserted into a corresponding one of each of the plurality of housing units. Each of the plurality of interdental devices may be interlocked with a corresponding one of the plurality of housing units, where each of the plurality of interdental devices includes a cap at a top portion configured to interlock and seal with the corresponding one of the plurality of housing units, and an applicator tip at a bottom portion configured to apply the predetermined amount of the material to an interdental space. The integrated pack of single-use oral delivery devices may be formed by breakable interconnections interspersed between each of the plurality of housing units and/or between each of the plurality of interdental devices.

In still another aspect of the present disclosure, a method of forming an integrated pack of single-use oral delivery devices includes forming a plurality of housing units, filling each of the plurality of housing units with a predetermined amount of a material, forming a plurality of filled housing units, and inserting one of a plurality of interdental devices into a corresponding one of each of the plurality of filled housing units. Each of the plurality of interdental devices may comprise a cap at a top portion for interlocking and sealing with a corresponding one of the plurality of filled housing units, and an applicator tip at a bottom portion for applying the predetermined amount of the material to the interdental space. The method further includes interlocking each of the plurality of interdental devices with a corresponding one of the plurality of filled housing units.

Various embodiments of the present disclosure provide an oral delivery device for the health of the teeth, gums and general health, as well as delivery of a fresh taste and odorless oral cavity. The oral delivery device may be used for treatment of sensitive teeth, dry mouth, dental decay, dental carry, gingivitis, sensitive teeth, dry mouth, oral sores, ulcer, cancer and/or fungus in the mouth. Use of the oral delivery device described herein, wherein the material to be applied contains a base of alkaline minerals or substances, not only slows or stops the tooth cavity process but also remineralizes the gum and teeth and can reverse the dental decay process, particularly after meals and in the evening time. At the same time, the oral delivery device while delivering active ingredients may also be used to massage the gums and clean the interdental space of debris and bacteria.

These as well as other aspects, advantages, and alternatives will become apparent to those of ordinary skill in the art by reading the following detailed description with reference where appropriate to the accompanying drawings. Further, it should be understood that the description provided in this summary section and elsewhere in this document is intended to illustrate the claimed subject matter by way of example and not by way of limitation.

### BRIEF DESCRIPTION OF THE DRAWINGS

The illustrated embodiments of the subject matter will be best understood by reference to the drawings, wherein like parts are designated by like numerals throughout. The following description is intended only by way of example, and simply illustrates certain selected embodiments of devices, systems, and processes that are consistent with the subject matter as claimed herein:

FIG. 1A illustrates an oral delivery device, in accordance with one or more embodiments of the present disclosure;

FIG. 1B illustrates a housing unit of the oral delivery device, in accordance with one or more embodiments of the present disclosure;

FIG. 1C illustrates an interdental device of the oral delivery device, in accordance with one or more embodiments of the present disclosure;

FIG. 2 illustrates application of material to the interdental space of the user, in accordance with one or more embodiments of the present disclosure;

FIG. 3 illustrates an interdental device, in accordance with one or more embodiments of the present disclosure; and

FIGS. 4A-4D illustrate an integrated pack of oral delivery devices, in accordance with one or more embodiments of the present disclosure.

### DETAILED DESCRIPTION OF THE EXEMPLARY EMBODIMENTS

Example apparatus are described herein. Other example embodiments or features may further be utilized, and other changes may be made, without departing from the spirit or scope of the subject matter presented herein. In the following detailed description, reference is made to the accompanying drawings, which form a part thereof.

The example embodiments described herein are not meant to be limiting. It will be readily understood that the aspects of the present disclosure, as generally described herein, and illustrated in the drawings, can be arranged, substituted, combined, separated, and designed in a wide variety of different configurations, all of which are explicitly contemplated herein.

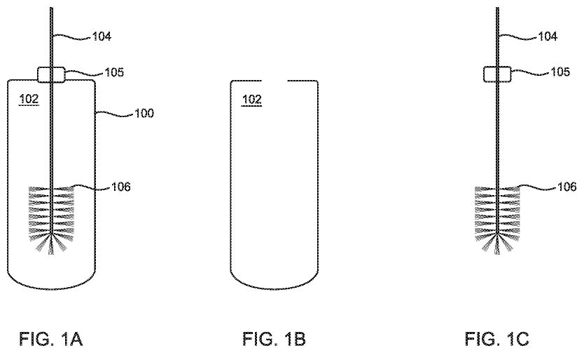

FIG. 1A illustrates an oral delivery device 100, in accordance with one or more embodiments of the present disclosure. In one or more embodiments, the oral delivery device 100 is a single-use device that is formed of a housing unit 102, and an interdental device 104 interlocked with the housing unit 102.

The housing unit 102 (as illustrated independently with reference to FIG. 1B) is configured to house a predetermined amount of oral medication, active agent or other material to be applied to the gum and interdental space of a user. In one or more embodiments of the present disclosure, the housing unit 102 may house the material in the form of a liquid, gel, paste, cream, or powder, or any combination thereof, containing active ingredients and/or pharmaceutical ingredient for release and/or sustained release in a specific dosage, which may also contain antiseptic/antibacterial solution for oral hygiene. In some embodiments, the material may have a pH of between 6.2 and 7.6.

Examples of the oral medication include, but are not limited to, benzocaine, nitroglycerine, aspirin, ibuprofen, diabetes medication, stimulants, relaxants, pain killers, herbal medications, plaque dissolving agents, anti-microbial and anti-bacterial agents, antibiotics, allergy medication, numbing agents, agents for sensitive teeth, dry mouth, oral cancer and oral sores, periodontal and gum disease treatments, and tooth-whitening agents. Examples of the tooth-whitening agents include, but are not limited to hydrogen peroxide and carbamide peroxide. Examples of the agents for sensitive teeth include, but are not limited to, potassium nitrate, Stannous flouride, flouride, strontium chloride. Examples of dry mouth or xerostomia treatments include but are not limited to, artificial saliva, Saliva inducing agents or medication such as pilocarpine, cevimeline, xylitol, carboxymethyl cellulos, hydroxyethyl cellulos, hemuctants, lubricants, moisturizers, herbal sialagogues, lactoperoxidase, lysozyme, glucose oxidase, lactoferrin and glycerol. Examples of other active ingredients include but are not limited to coenzyme Q10, resveratrol, acetaminophen, melatonin, metronidazole, sodium fluoride, testosterone, terbutaline sulphate, benzocaine, chlorhexidine, doxycycline, metronidazole, collagenase, prostaglandin E2, minocycline, amoxicillin, naproxen sodium, doxylamine, fluoride, naproxen, nicotine, minerals including pH ion alkaline minerals (calcium, magnesium, potassium, manganese, iron, phosphorus), antioxidants, vitamins (including but not limited to vitamin A, B, C, D, E, K), Cannabidiol (CBD) and tetrahydrocannabinol (THC). As a feature of the oral delivery device 100, the oral medication may be soluble in the user's saliva for easy ingestion thereinto and/or be absorbed directly through the buccal cavity to the blood stream. In another embodiment of the present disclosure, the oral medication may include a sweetener. In yet another embodiment of the present disclosure, the oral medication may include salivary enzymes to help neutralize odors, inhibit harmful bacteria, and remove tartar and/or plaque. In any event, it should be appreciated that the oral delivery device 100 of the present disclosure may be configured to contain and apply any form of oral medication, vitamins, minerals, active agent or other material to the gum and interdental space of a user.

Although, the housing unit 102 is shown to be cylindrical in shape, it would be apparent to one of ordinary skill in the art that the housing unit 102 may be formed of various other shapes such as round, circular, triangle, oval, octagonal, trapezoid, parallelogram, rhombus, rectangle, hexagonal and square shapes.

The interdental device 104 (as illustrated independently with reference to FIG. 1C) includes a cap 105 at a top portion for interlocking and sealing with the housing unit 102, and an applicator tip 106 at a bottom portion for applying medication, active agent or other material to the interdental space of the user. In an example, the applicator tip 106 is in form of a modern toothpick. In another example, the applicator tip 106 is in form an interdental brush to locally supply an approximate dose of the oral medication, active agent or other material to the user.

The interlocking and sealing between the cap 105 and the housing unit 102 may be achieved through a variety of shapes, designs and sealing mechanisms. Example of the sealing mechanism include, but is not limited to, a snap-fit arrangement in which the housing unit 102 may include a snap-fit bottle neck designed for the cap 105 to snap fit thereonto. The snap-fit lock may be easily opened or broken for immediate application of the material contained in the housing unit 102. In one or more embodiments of the present disclosure, the locking and sealing of the cap 105 with the housing unit 102 keeps the material contained in the housing unit 102 clean and fresh, until it is ready for use.

In one or more embodiments of the present disclosure, the housing unit 102 and the interdental device 104 may be molded from a variety of rigid materials, including plastics, resins, etc., such as, for example, polypropylene. The softness/rigidity of the interdental device 104 may be tailored by various polymer compositions and the geometric structure.

In one or more embodiments of the present disclosure, the applicator tip 106 may be medicated with an additive such as a breath freshener, a mint flavor, a citrus flavor, a clove flavor, a cinnamon flavor, a berry flavor, or other desirable flavors to mask the taste of the medication, enhance the experience or for personal hygiene purposes. In another embodiment of the present disclosure, the medicated interdental device 106 may be used as an independent product to deliver such flavor, vitamins and minerals to the user. As a result of these novel features, the combination of the interdental device 104 and the housing unit 102 is an effective way to encourage the absorption of vitamins, minerals and/or medication either locally to the gums, teeth, dental roots, or systemically through the buccal cavity and/or ingestion.

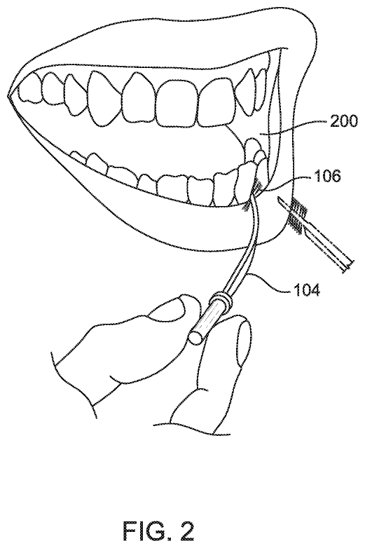

With reference now to FIG. 2, one example of the application of material (e.g., oral medication, active agent, etc.) to an interdental space 200 of a user, with help of the interdental device 104, is illustrated. In one or more embodiments of the present disclosure, the applicator tip 106 may be made from soft low-density polyethylene (LDPE) or other elastomeric or felt materials, and may function as a gum protector with low mechanical irritation, yet, durable enough to remove inter-tooth food leftovers and contaminations.

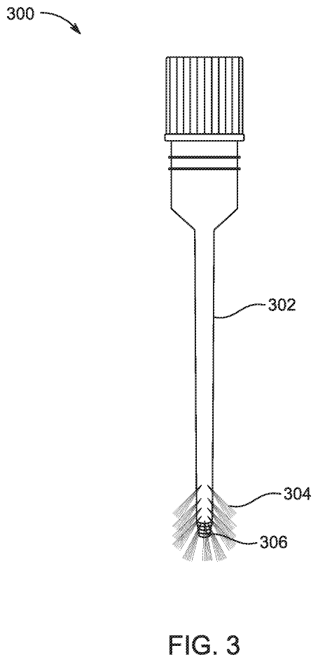

FIG. 3 illustrates an interdental device 300 in accordance with one or more embodiments of the present disclosure. The interdental device 300 includes an inner rigid stick like portion 302, and a softer outer layer 304. The softer outer layer 304 may be formed of various materials, examples of which include, but not limited to, various forms of plastics, polyethylene, silicone, and other polymers. The softer outer layer 304 may be made of felt, or felt like material to form an applicator tip similar to the applicator tip of FIG. 1C.

In one or more embodiments of the present disclosure, the interdental device 300 may be manufactured using co-injection molding of the inner rigid stick like portion 302, and the softer outer layer 304.

In one or more embodiments of the present disclosure, the interdental device 300 includes one or more interconnected pores 306 of micro or nano-size open cells. The open pore structure 306 may be made of various polymers including a wide range of polymers from hydrophobic polymer such as polypropylene (PP) to relatively hydrophilic polymer such as polyamide-6 (Nylon 6) foam.

In one or more embodiments of the present disclosure, the open pore structure 306 enables the interdental device 300 to release said quantity of active ingredient in a time substantially equivalent to using device 300. The time necessary to ingest the active ingredient corresponds substantially with the time required for tooth cleaning using the interdental device 300. In another embodiment of the present disclosure, the open pore structure 306 is configured for prolonged release of the selected material even after the immediate release of the material from a corresponding housing unit. The interdental device 300 may concentrate the selected material (e.g., oral medication, active agent, etc.) at a bottom end 306 so that the remaining portion of the device 300 is outside of the mouth.

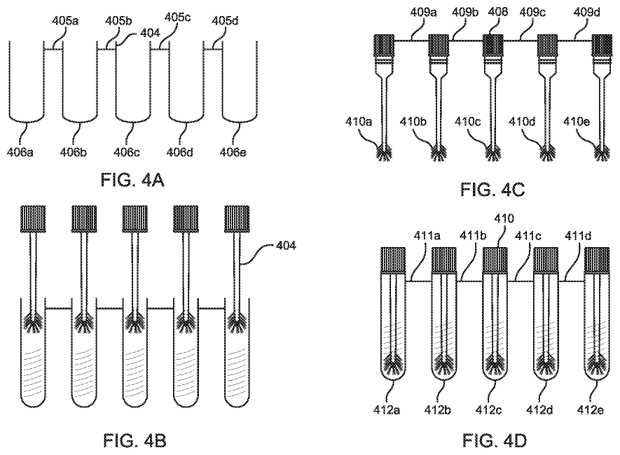

FIGS. 4A-4D illustrate the formation of an integrated pack of oral delivery devices 100, in accordance with one or more embodiments of the present disclosure.

As illustrated in FIG. 4A, an integrated pack 404 of empty housing units 406a-406e is first formed by a manufacturer. Each of the empty housing units 406a-406e may be similar to the housing unit 102 described with reference to FIG. 1B. Although, the integrated pack 404 is shown to include five housing units 406a-406e, it would be apparent to one of ordinary skill in the art that the integrated pack 404 may be comprised of a plurality of housing units that is more or less than five. In one or more embodiments, the integrated pack 404 may be manufactured in accordance with an injection molding process such that each of the empty housing units 406a-406e are attached to each other during the manufacturing process by interconnections 405a-405d. It should be appreciated that interconnections 405a-405d are configured to be readily breakable by the user so that, once fully manufactured, each oral delivery device may be easily detachable from the integrated pack 404. It should be appreciated that the interconnections 405a-405d may be formed of polypropylene or other polymer, thereby facilitating such easy detachment by the user.

Thereafter, as illustrated with reference to FIG. 4B, the integrated pack 404 of empty housing units 406a-406e may then be filled with a predetermined amount of selected material (e.g., oral medication, active agent, etc.). The filling of the empty housing units 406a-406e may be done simultaneously or in parallel, or one by one, during the pack manufacturing process.

Thereafter, as illustrated with reference to FIG. 4C, an integrated pack 408 of interdental devices 410a-410e may be separately formed during the pack manufacturing process. Each of the interdental devices 410a-410e may be similar to the interdental device 104 described with reference to FIG. 1C. The pack 408 may include a number of medicated or mineralized toothpicks, and a number of toothpick-shaped devices. As with the housing units 406a-406e of FIG. 4A, the interdental devices 410a-410e may be similarly attached to each other by way of easily breakable interconnections 409a-409d. Alternatively, only one of the housing units 406a-406e or interdental devices 410a-410e need be formed into an integrated pack, as for example shown in FIG. 4D.

Referring now to FIG. 4D, the interdental devices 410a-410e (whether formed into an integrated pack 408 or otherwise) may then be inserted into corresponding housing units 406a-406e (whether formed into an integrated pack 404 or otherwise) and locked thereonto using a snap-fit arrangement to form an integrated pack 410 of oral delivery devices 412a-412e. As noted above, only one of the housing units 406a-406e or interdental devices 410a-410e need be formed into an integrated pack such that the final oral delivery devices 412a-412e are formed into an integrated pack 410 by virtue of at least one set of interconnections 411a-411d. While the interconnections 411a-411d of FIG. 4d are shown as being between the housing units (such as the case in FIG. 4A), it should equally be appreciated that the interconnections 411a-411d of FIG. 4d may instead (or in addition to) be formed between the interdental devices (such as the case in FIG. 4C).

The interdental devices 410a-410e may cap the housing units 406a-406e respectively. The final integrated pack 410 is ready to be sold into the market, wherein each of the oral delivery devices 412a-412e is a single-use product, and is joined with each other using a weak connection, so that each single-use product can be disconnected easily for quick use. In one or more embodiments of the present disclosure, each of the oral delivery devices 412a-412e is a single-use medicated or mineralized unit that may be marked or packaged with a medicated or mineralized interdental device with a precise dosage. The selected material (e.g., oral medication, active agent, etc.) in the oral delivery devices 412a-412e remain sealed until the end user is ready to use the product.

Various embodiments of the present disclosure provide for simple and cost effective fabrication based injection molding and double injection molding of multiple, interconnected single-use devices. Moreover, each single-use unit 412a-412e is prefilled with a pre-determined amount of material (e.g., drugs, vitamins, minerals, etc.) such that the consumer will know the exact dosage provided.

It will be appreciated that variants of the above-disclosed and other features and functions, or alternatives thereof, may be combined into many other different systems or applications. Various presently unforeseen or unanticipated alternatives, modifications, variations, or improvements therein may be subsequently made by those skilled in the art which are also intended to be encompassed by the following claims.

What is claimed is:

1. A single-use oral delivery device comprising:
a housing unit configured to house a predetermined amount of a material to be applied to an interdental space of a user; and
an interdental device configured to be interlocked with the housing unit, wherein the interdental device includes a cap at a top portion configured to interlock and seal with the housing unit, and an applicator tip at a bottom portion configured to apply the predetermined amount of the material to the interdental space.

2. The single-use oral delivery device of claim 1, wherein the housing unit houses the material in the form of at least one of: liquid, gel, paste, cream, and powder.

3. The single-use oral delivery device of claim 2, wherein the material is characterized by a pH of between 6.2 and 7.6.

4. The single-use oral delivery device of claim 1, wherein the material is selected from at least one of: minerals, pH ion alkaline minerals, vitamins, benzocaine, nitroglycerine, aspirin, ibuprofen, diabetes medication, stimulants, relaxants, pain killers, herbal medications, plaque dissolving agents, anti-microbial and anti-bacterial agents, anti-biotics, allergy medication, numbing agents, agents for sensitive teeth, dry mouth, agents for the treatment of xerostomia, gingivitis, cancer, oral sores, tooth-whitening agents, breath fresheners and taste agents.

5. The single-use oral delivery device of claim 1, wherein the applicator tip is in form of a modern toothpick or an interdental brush.

6. The single-use oral delivery device of claim 1, wherein the applicator tip is made from a material selected from at least one of: low-density polyethylene (LDPE), elastomeric material, and felt material.

7. The single-use oral delivery device of claim 1, wherein the cap interlocks with a neck of the housing unit through a snap-fit arrangement.

8. The single-use oral delivery device of claim 1, wherein the applicator tip is medicated with an additive selected from at least one of: a breath freshener, a mint flavor, a citrus flavor, a clove flavor, a cinnamon flavor, and a berry flavor.

9. The single-use oral delivery device of claim 1, wherein the interdental device comprises:
an inner rigid portion extending vertically along the housing unit; and
a soft outer layer at a bottom end of the inner rigid portion, wherein the soft outer layer forms the application tip.

10. The single-use delivery device of claim 9, wherein the inner rigid portion has a bottom end that includes an open pore structure made of various polymers selected from at least one of: hydrophobic polymer such as polypropylene (PP) and hydrophilic polymer such as polyamide-6 (Nylon 6) foam.

11. The single-use oral delivery device of claim 1, wherein the material to be released is configured to slowly release the active ingredients over a period of a time thereby prolonging the therapeutic effects of the material applied.

12. An integrated pack of single-use oral delivery devices comprising:
a plurality of housing units each containing a predetermined amount of a material; and
a plurality of interdental devices inserted into a corresponding one of each of the plurality of housing units,
wherein each of the plurality of interdental devices is interlocked with a corresponding one of the plurality of housing units, wherein each of the plurality of interdental devices includes a cap at a top portion configured to interlock and seal with the corresponding one of the plurality of housing units, and an applicator tip at a bottom portion configured to apply the predetermined amount of the material to an interdental space,
wherein the integrated pack of single-use oral delivery devices is formed by breakable interconnections interspersed between each of the plurality of housing units and/or between each of the plurality of interdental devices.

13. The integrated pack of single-use oral delivery devices of claim 12, wherein the material in the form of at least one of: liquid, gel, paste, cream, and powder.

14. The integrated pack of single-use oral delivery devices of claim 13, wherein the material is characterized by a pH of between 6.2 and 7.6.

15. The integrated pack of single-use oral delivery devices of claim 12, wherein the applicator tip of each of the plurality of interdental devices is in form of a modern toothpick or an interdental brush.

16. The integrated pack of single-use oral delivery devices of claim 12, wherein the cap of each of the plurality of interdental devices interlocks with a neck of a corresponding one of the plurality of housing units through a snap-fit arrangement.

17. A method of forming an integrated pack of single-use oral delivery devices, the method comprising:
forming a plurality of housing units;
filling each of the plurality of housing units with a predetermined amount of a material, forming a plurality of filled housing units;
inserting one of a plurality of interdental devices into a corresponding one of each of the plurality of filled housing units, wherein each of the plurality of interdental devices comprises a cap at a top portion for interlocking and sealing with a corresponding one of the plurality of filled housing units, and an applicator tip at a bottom portion for applying the predetermined amount of the material to the interdental space;
interlocking each of the plurality of interdental devices with a corresponding one of the plurality of filled housing units.

18. The method of claim 17, further comprising forming the integrated pack of single-use oral delivery devices by connecting the plurality of housing units to each other by breakable interconnections interspersed therebetween.

19. The method of claim 17, further comprising forming the integrated pack of single-use oral delivery devices by connecting the plurality of interdental devices to each other by breakable interconnections interspersed therebetween.

20. The method of claim 17, wherein the applicator tip is in form of a modern toothpick or an interdental brush.

21. The method of claim 17, wherein each interdental device comprises:
an inner rigid portion extending vertically along the housing unit; and
a soft outer layer at a bottom end of the inner rigid portion, wherein the soft outer layer forms the application tip.

22. The method of claim 21, wherein the inner rigid portion has a bottom end that includes an open pore structure that enables corresponding interdental device to release a predetermined quantity of the oral medication in a time substantially equivalent to using the interdental device, wherein the open pore structure is configured to enable a prolonged release of the oral medication after an immediate release of medication from corresponding housing unit.
